(19) United States
(12) Patent Application Publication
(10) Pub. No.: US 2009/0324660 A1
(43) Pub. Date: Dec. 31, 2009

(54) PERSONAL-CARE ARTICLE FOR SEQUENTIALLY DISPENSING COMPOSITIONS WITH DIFFERENT FRAGRANCES

(76) Inventors: Jonathan Robert Cetti, Mason, OH (US); Christopher Dean Putman, West Chester, OH (US); Andres Ernesto Velarde, Cincinnati, OH (US); Karen Lehnhoff, Cincinnati, OH (US); Edward Dewey Smith, III, Mason, OH (US); Julien Claude Plos, Cincinnati, OH (US); Timothy Alan Scavone, Loveland, OH (US); Steven Edward Witt, Harrison, OH (US); Joyce Marie Benjamin, Mason, OH (US); Cynthia Louise Crisp, Fairfield, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/361,474

(22) Filed: Jan. 28, 2009

Related U.S. Application Data
(63) Continuation-in-part of application No. 11/881,535, filed on Jul. 27, 2007.

(51) Int. Cl. A61K 8/28; A61K 8/02; A61Q 15/00; A61Q 19/10

(52) U.S. Cl. 424/401; 424/66; 424/65; 510/146

(57) ABSTRACT

The present invention relates to a personal-care article for dispensing and or applying a personal-care product that comprises a package and a personal-care product. The package comprises at least one chamber, a dispensing orifice, a first zone proximate to the dispensing orifice and a second zone distal to the dispensing orifice, wherein the first zone and the second zone are both located in the at least one chamber. The personal-care product comprises a first personal-care composition substantially disposed within the first zone and the second personal-care composition substantially disposed within the second zone. The first composition comprises a first fragrance releaseably associated with a suppressing agent and the second composition comprises a second fragrance releaseably associated with a suppressing agent; wherein the first fragrance is different from the second fragrance.

### CROSS REFERENCE TO RELATED APPLICATION

This application is a continuation-in-part of U.S. application Ser. No. 11/881,535, filed Jul. 27, 2007, incorporated herein by reference in its entirety.

### FIELD OF THE INVENTION

The present invention relates to a personal-care article comprising a package and a personal-care product comprising at least two compositions each having a fragrance which is different from the other.

### BACKGROUND OF THE INVENTION

Personal-care products are well known and widely used for providing numerous benefits, such as, for example, cleansing and moisturizing skin and hair, delivering actives, hiding imperfections, reducing oiliness/shine, controlling body odor, as well as, providing scent to the hair and/or the skin. The efficacy of these types of products is directly related to the frequency of use. Consumers often habituate or tire of a particular scent of a personal-care product over time. When this habituation occurs, consumers often decrease or even or stop use of one personal-care product and begin to use another personal-care product with another scent despite the benefits gained by compliant use of the first personal-care product over time. With space in the bathroom, vanity, shower, or bath being limited, a typical consumer does not have enough space to store multiple containers of personal-care products to meet the consumers' need for different scents to prevent scent habituation. There is a need to provide a personal-care product that is able to differ in perceived scent over time.

Further, scent habituation may occur in personal-care products that have suppressed fragrances. Theses products comprise fragrances or scents that may not be noticeable before use but can become noticeable either when the personal care product is applied, or appear after the passage of time or in response to an activation mechanism (e.g., via a sweat event). There is a desire to provide personal-care products having two or more suppressed fragrances. A challenge with incorporating more than one suppressed fragrance is keeping the fragrances stable in the preferred section of the product. Accordingly, there is also a desire to create personal-care products having different fragrances wherein the fragrances are stable and do not migrate into other sections of the product.

### SUMMARY OF THE INVENTION

The present invention relates to a personal-care article that comprises a package and a personal-care product. The package comprises at least one chamber, a dispensing orifice, a first zone proximate to the dispensing orifice, and a second zone distal to the dispensing orifice, wherein said first zone and said second zone are both located in the at least one chamber. The personal-care product comprises a first personal-care composition substantially disposed within the first zone and a second personal-care composition substantially disposed within the second zone. The first composition comprises a first fragrance and the second composition comprises a second fragrance; wherein the first fragrance is different from the second fragrance. The first and second fragrances can be neat fragrances, fragrances associated with a suppressing agent, or a combination of the two. Thus, the personal-care product dispensed from the package changes in fragrance over the product lifetime, thereby overcoming the problem of consumer habituation to a scent. In one exemplary embodiment, the first and second compositions are in direct physical contact with each other. In another exemplary embodiment, the first and second compositions are separated by a consumer-removable barrier member.

### BRIEF DESCRIPTION OF THE DRAWINGS

FIG. 1 illustrates a personal-care article having two zones, each zone comprising one fragrance.

FIG. 2 illustrates a personal-care article having two zones, each zone comprising two fragrances.

FIG. 3 illustrates a personal-care article having two zones, each zone comprising two fragrances, and a diagonal interface between zones.

FIG. 4 illustrates a personal-care article having two zones, each zone comprising two fragrances.

FIG. 5 illustrates a personal-care article having two zones, each zone comprising three fragrances.

FIG. 6 illustrates a personal-care article having two zones, each zone comprising one fragrance.

FIG. 7 illustrates a personal-care article having three zones, each zone comprising one fragrance.

FIG. 8 illustrates a personal-care article having three zones, the first and second zones each comprising two fragrances, the third zone comprising one fragrance.

FIG. 9 illustrates a personal-care article having three zones, each zone comprising two fragrances.

FIG. 10 illustrates a personal-care article having three zones, each zone comprising two fragrances.

FIG. 11 illustrates a cross-section of a personal-care article having two zones, wherein Zone 2 is contained within Zone 1.

FIG. 12 illustrates a chart rating consumers' anticipation of a change in fragrance over the time of usage.

### DETAILED DESCRIPTION OF THE INVENTION

The articles and products of the present invention can comprise, consist of, or consist essentially of the essential features described herein, as well as any of the additional or optional ingredients, components, or limitations described herein useful in personal-care compositions.

The term “ambient conditions” as used herein, refers to surrounding conditions at one (1) atmosphere of pressure, 50% relative humidity, and 25° C.

The term “personal-care product,” as used herein, may include, but is not limited to: antiperspirants, deodorants, lotions (e.g. hand lotion and body lotion), skin-care products (e.g., face and neck lotions, serums, sprays), sunless tanners, cosmetics (e.g., foundation, concealer, blush, lipstick, lip gloss), depilatories, shampoos, conditioning shampoos, hair conditioners, body washes, moisturizing body washes, shower gels, skin cleansers, cleansing milks, hair and body washes, in-shower body moisturizers, pet shampoos, shaving preparations, after-shaves, razor moisturizing/lubricating strips, razor shave-gel bars, bar soaps, cleansing products, feminine-care products, oral-care products, and baby-care products.

The term “personal-care composition,” as used herein, refers to compositions intended for topical application to the body. Compositions of the present invention may be leave-on formulations—in which the product is applied topically to the skin, hair or teeth, for example, and left on for a period of time, or rinse-off formulations—in which the product is applied topically to the skin or hair and then is subsequently rinsed within minutes from the skin, hair or teeth with water, or otherwise wiped off using a substrate with deposition of a portion of the composition. The personal-care composition of the present invention is typically extrudable or dispensible from a package. In other embodiments, the personal-care article may not comprise a package at all—for instance, in the case of bar soap. The personal-care compositions of the present invention can be in any form, including, for example, solid, semi-solid, liquid, semi-liquid, cream, lotion or gel compositions intended for topical application to the body.

The term “package,” as used herein, includes any suitable container for a personal-care composition such as, for example, a canister, bottle, tottle (bottle which rests on its closure), tube, jar, aerosol container, pressurized containers, non-aerosol pump and mixtures thereof.

The term “dispensing orifice,” as used herein, refers to any opening in a package through which product may be dispensed and/or applied. For example, an antiperspirant package may comprise a dispensing orifice in the form of a perforated or mesh-like application surface. The dispensing orifice may be the large opening in the end of a canister; for example, those that hold antiperspirant or deodorant sticks.

As used herein the term “zone,” as used herein, is a domain or region within a package which corresponds to a composition of the personal-care product. A zone within a package may be in direct physical contact with another zone within a package, such that the compositions corresponding to the zone are in direct physical contact with one another. An interface between the zones can be distinct or gradual or separated by another zone. The amount of composition contained in a zone can be defined by a percentage of the package volume, as discussed later.

The term “fragrance,” as used herein, refers to formulations or compositions made up of one or a mixture of perfume chemicals/raw materials and/or odor-controlling materials. Differences between fragrances may result from same chemical make-ups (presence and concentration of individual perfume chemicals/raw materials and/or odor-controlling materials) but different overall concentrations, same overall concentrations but different chemical make-ups, different chemical make-ups and different overall concentrations, and the like.

All percentages, parts and ratios are based upon the total weight of the compositions of the present invention, unless otherwise specified. All such weights as they pertain to listed ingredients are based on the active level and, therefore; do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about.” All molecular weights as used herein are weight average molecular weights expressed as grams/mole, unless otherwise specified.

The present invention relates to a personal-care article for dispensing and applying a personal-care product. In a preferred embodiment, the article comprises a package and a personal-care product.

### I. Package

In one embodiment, the package comprises at least one chamber, a dispensing orifice, a first zone proximate to the dispensing orifice, and a second zone distal to the dispensing orifice. The package may comprise a first end, a second end, a vertical axis running from the first end to the second end, wherein the dispensing orifice is located at the first end. In a preferred embodiment, the first zone and second zone are located in one chamber. In an alternative embodiment, the first zone and second zone are located in two or more chambers.

The number of zones within a package, and thus the number of personal-care compositions disposed within the respective zone can vary in number. For example, the package may have three zones and three personal-care composition within the respective zones; four zones and four compositions, five zones and five compositions, and so on. In preferred embodiments, a zone within a chamber is in direct physical contact with another zone within the same chamber, such that the compositions corresponding to the zone are in direct physical contact with one another. In an alternative embodiment, a removable barrier member is placed between adjacent compositions. The interface between the zones can be distinct or gradual.

In one embodiment, the package comprises two zones and the first zone is in physical contact with the second zone within the package. In another embodiment, the package comprises a third zone, medial to the dispensing orifice, which separates the first and second zones within the package. The third zone may comprise a third personal-care composition substantially disposed within the third zone which may or may not have a fragrance. If the third composition has a fragrance, it may be different from the first fragrance and/or the second fragrance. The term “substantially disposed” as used herein, means that the personal-care composition is primarily located in a particular zone. In some embodiments, there may be some diffusion or blending of compositions between zones. In particular embodiments, the composition of the third zone may comprise a blend of the compositions of the first and second zones. In other embodiments, the composition of the third zone may be different from the compositions of the first and second zones.

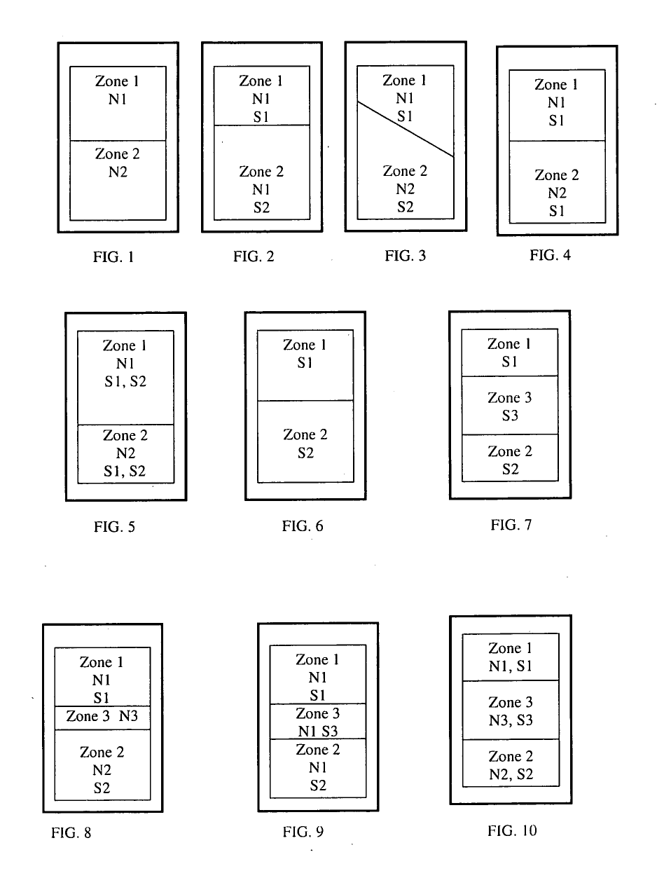

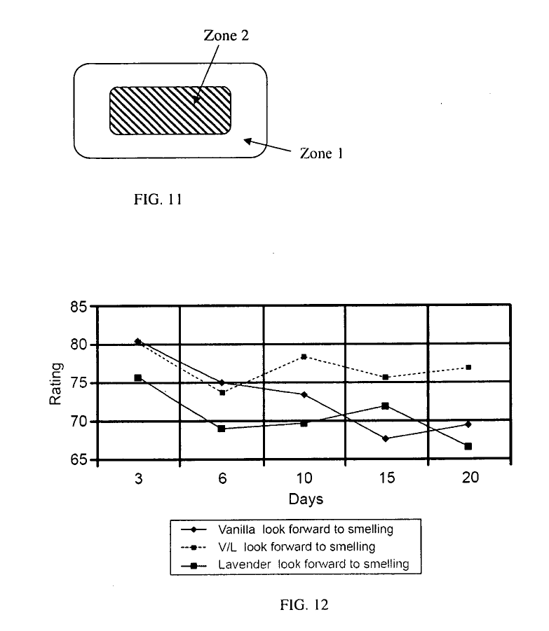

In one embodiment, such as shown in FIG. 1, the first zone and the second zone comprise a substantially equal percentage, by volume, of the package (e.g., first zone comprises 50%, second zone comprises 50%; first zone comprises 45%, second zone comprises 55%). In another embodiment, such as shown in FIG. 7, the first zone, the second zone and the third zone comprise a substantially equal percentage, by volume, of the package (e.g., first zone comprises 33%, second zone comprises 33%, third zone comprises 33%; first zone comprises 30%, second zone comprises 30%, third zone comprises 40%). In further embodiments, the volume percentage of the zones may not be equal (e.g., 5% difference, 10% difference, 20% difference). For instance, as shown in FIG. 8, the first and second zones may be equal volume percentages, and the third zone may be a smaller volume percentage (e.g., first zone comprises 40%, second zone comprises 40%, third zone comprises 20%; first zone comprises 45%, second zone comprises 45%, third zone comprises 5%). In still further embodiments, the first and second zones may be separated by a very thin third zone.

### II. Personal-care Product

The personal-care product comprises a first personal-care composition substantially disposed in the first zone and a second personal-care composition substantially disposed in the second zone. The first composition comprises a first fragrance, the second composition comprises a second fragrance, and the first fragrance is different from the second fragrance. The respective fragrances of the personal-care compositions remain substantially distinct although the compositions are contained within the same chamber. Generally, the first personal-care composition is substantially dispensed prior to dispensing the second composition, so that there is a limited amount of mixing of the fragrances of the first composition with that of the second composition. The term “substantially dispensed” as used herein, unless otherwise specified, means that at least 10%, or at least 25%, or at least 50% of said first personal-care composition substantially disposed within said first zone is dispensed prior to the dispensing of the second personal-care composition substantially disposed within the second zone. An example of this sequential dispensing: a package may dispense a vanilla scented personal-care composition, followed by a lavender scented composition, followed by a vanilla scented personal-care composition. Thus, the personal-care product changes in fragrance as it is dispensed from the package which overcomes the problem of consumer habituation to scent. If there are more than two zones of personal-care compositions, in a preferred embodiment, the first composition is substantially dispensed prior to dispensing the second composition, the second composition is substantially dispensed prior to dispensing the third composition, etc.

In a preferred embodiment, the personal-care product is an antiperspirant or deodorant. Many types of antiperspirants are commercially available or otherwise known in the art. Some of these products contain an antiperspirant active dispersed within a suitable carrier, and optionally a solid gellant or wax matrix that provides the product with a desired consistency or hardness.

In another embodiment, the personal-care product is a personal-care bar that can be used alone or in connection with an implement, such as, for example, a razor. The personal-care bar product comprises a horizontal axis, a vertical axis, and a center where the horizontal axis and vertical axis cross. The product further comprises a first zone distal to the center and a second zone proximate to the center so that the first zone surrounds the second zone. A first personal-care composition is substantially disposed in the first zone, and a second personal-care composition is substantially disposed in the second zone. The first composition comprises a first fragrance and the second composition comprises a second fragrance; the first fragrance is different from the second fragrance. In a preferred embodiment, the first composition is a different hue from the second composition.

Personal-care products of the present invention comprise compositions having two or more fragrances selected from neat fragrances and suppressed fragrances. It is preferred to have different fragrances in different zones of compositions. The challenge with incorporating more than one fragrance is keeping the fragrances stable in the preferred zone.

The term “stable” as used herein, unless otherwise specified, means that the compositions of the personal-care product maintain at least two “separate” compositions when sitting in physical contact at ambient conditions for a period of at least 1 week according to the dialysis method described hereinafter. By “separate,” it is meant that there is substantially no mixing of at least two fragrances in compositions proximate to each other, such that less than 30% of the concentration of at least one fragrance of interest within the first composition migrates to the second composition or vice versa.

A. Neat Fragrances

Personal-care articles of the present invention may comprise a neat fragrance comprising any perfume or perfume chemical suitable for topical application to the skin. In a preferred embodiment, antiperspirant compositions comprise neat fragrances to help cover or mask malodors resulting from perspiration, or which otherwise provide the compositions with the desired perfume or unscented/neutral aroma.

The concentration of the neat fragrance in the antiperspirant compositions should be effective to provide the desired aroma. As used herein, “unscented” refers to the level of fragrance wherein the level of fragrance is below 0.05% such that the fragrance is absent or undetected. Generally, the concentration of the scented neat fragrance is from at least about 0.05%, from about 0.1%, from about 0.5% but no more than about 20%, no more than about 10%, no more than about 5%, or no more than about 2%, by weight of the composition. The neat fragrance should not impart excessive stinging to the skin, especially broken or irritated skin, at the concentrations disclosed herein. The neat fragrance may be included in the antiperspirant compositions of the present invention as a free perfume.

The neat fragrance may include perfumes/aromatic materials known to a person of ordinary skill in the art of creating fragrances. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

B. Suppressed Fragrances

Personal care compositions of the present invention may additionally or alternatively employ a suppressed fragrance. This type of fragrance utilizes a suppressing agent associated with the perfume molecules to inhibit its expression until activated by one or more mechanisms, such as through shear during application of the compositions or through a sweat event or other bodily fluid interaction. A suppressing agent may be employed in the antiperspirant or deodorant product, wherein the suppressing agent comprises a means of attaching to the fragrance to prohibit the scent from being immediately perceived by a consumer. Suppressing agents may limit natural diffusion of and/or immobilize fragrances. This encourages stability of fragrances in a given zone. Exemplary fragrance suppressing agents include, but are not limited to, starches, oligosaccharides (e.g., cyclodextrins), polyethylenes, polayamides, polystyrenes, polyisoprenes, polycarbonates, polyesters, polyacrylates, vinyl polymers, silicas, and aluminosilicates. Commercially available encapsulating materials N-Lok™, manufactured by National Starch, Narlex™ (ST and ST2), and Capsul E™ are useful for the present invention. These materials comprise pregelatinized waxy maize starch and optionally, glucose. The starch is modified by adding monofunctional substituted groups such as octenyl succinic acid anhydride.

In some embodiments, there may not be a neat fragrance associated with the personal-care product, but there may be a suppressed fragrance. Such suppressed fragrance personal-care products may be marketed with the following terms: unscented, scent-free, sensitive, and/or hypoallergenic. In other embodiments, the intent is for the consumer to initially perceive a neat fragrance and not perceive (or only minimally perceive) a suppressed fragrance that is present. In either case, during application and/or use, or after a certain amount of time, the suppressed fragrance may be released. For example, a consumer may experience a woodsy neat fragrance when applying an antiperspirant product, but may not detect a suppressed ocean fragrance until it is triggered later on in the day. Perspiration or some other bodily trigger may release the suppressed fragrance enabling it to be perceived by the consumer. To accomplish this goal, a suppressing agent may be used. The suppressing agent may releaseably associate with the fragrance, thereby suppressing the scent from consumer detection until the fragrance is released upon activation.

The suppressed fragrance of the present invention may be substantially different and distinct from the neat fragrance in order to overcome the effect of fragrance habituation and to make the suppressed fragrance noticeable over the neat fragrance. Generally, the concentration of the suppressed fragrance is from at least about 0.05%, from about 0.1%, from about 0.5% but no more than about 20%, no more than about 10%, no more than about 5%, or no more than about 2%, by weight of the composition. The suppressed fragrance should not impart excessive stinging to the skin, especially broken or irritated skin, at the concentrations disclosed herein.

Personal-care products of the present invention may employ at least one suppressed fragrance. A representative, non-limiting, list of fragrance materials that may be suppressed with the suppressing agent includes anethole, benzaldehyde, decyl aldehyde, benzyl acetate, benzyl alcohol, benzyl formate, benzyl propionate, iso-bornyl acetate, camphene, cis-citral (neral), citronellal, citronellol, citronellyl acetate, paracymene, decanal, dihydrolinalool, dihydromyrcenol, methyl benzyl carbinyl acetate, dimethyl benzyl carbinyl acetate, dimethyl phenyl carbinol, eucalyptol, helional, geranial, geraniol, geranyl acetate, geranyl nitrile, cis-3-hexenyl acetate, dihydrocitronellal, d-limonene, linalool, linalool oxide, tetra-hydro linalool, alpha-methyl ionone, methyl nonyl acetaldehyde, methyl phenyl carbinyl acetate, menthyl acetate, laevo-menthyl acetate, menthone, iso-menthone, myrcene, myrcenyl acetate, myrcenol, nerol, neryl acetate, nonyl acetate, phenyl ethyl alcohol, phenyl acetaldehyde, alpha-pinene, beta-pinene, gamma-terpinene, terpineol, alpha-terpineol, beta-terpineol, terpinyl acetate, vertenex (para-tertiary-butyl cyclohexyl acetate), gamma-methyl ionone, undecalactone, undecylenic aldehyde, alpha-damascone, beta-damascone, amyl acetate, lemon oil, orange oil, and mixtures thereof.

Example combinations of personal-care articles with two to three zones and different combinations of fragrances are illustrated in FIGS. 1-10. In FIGS. 1-10, N1=a first neat fragrance, N2=a second neat fragrance, N3=a third neat fragrance, S1=a first suppressed fragrance, S2=a second suppressed fragrance, S3=a third suppressed fragrance. For example, FIG. 1 illustrates a personal-care article with two zones with a horizontal interface between the zones. Zone 1 comprises a first neat fragrance and zone 2 comprises a second neat fragrance. In a preferred embodiment, as shown in FIG. 2, the neat fragrances of zones 1 and 2 are the same, but the suppressed fragrances differ. In another example, FIG. 3 illustrates a personal-care article with two zones with a diagonal interface between the zones. Zone 1 comprises a first neat fragrance and a first suppressed fragrance, and Zone 2 comprises a second neat fragrance and a second suppressed fragrance. Embodiments are also envisioned where there are more than three zones.

C. Hues

In some embodiments, the personal-care compositions may comprise a dye, colorant, pigment, or the like, such that each personal-care composition is a different color or hue. Different hues in different compositions signal to the consumer that the product is multi-scented, that a fragrance shift occurs, or that there is a change in fragrance intensity. Additionally, the hue may be selected to represent the fragrance to enhance consumer delight. For example, if a watery fragrance, a blue or green hue may be selected; if a citrus fragrance, a yellow, orange, or lime green hue may be selected.

In one embodiment, a first personal-care composition can be a blue hue and a second personal-care composition can be a green hue. In another embodiment, a first personal-care composition can be a white hue and a second personal-care composition can be a pink hue. In another embodiment, a first personal-care composition can be a yellow hue, a second personal-care composition can be an orange hue, and a third personal-care composition can be a purple hue. The third composition may have a hue different from that of the first and second compositions, such that the consumer is informed that a fragrance shift occurs. For instance, first and second personal-care compositions can be a white hue and a third, medial composition can be a blue hue.

In further embodiments, suppressed fragrance particles may comprise a dye, colorant, pigment, or the like, such that each suppressed fragrance has a different color or hue. The fragrance itself, an encapsulation, etc. may have a hue. Many combinations are possible by varying the hues of the different compositions and the different suppressed fragrances. In one embodiment, a first personal-care composition can be a yellow hue and a second personal-care composition can be a yellow hue, but the first personal-care composition may comprise suppressed fragrance particles having a blue hue and the second personal-care composition may comprise suppressed fragrance particles having a green hue. In another embodiment, a first personal-care composition can be a yellow hue and a second personal-care composition can be a green hue, but the first personal-care composition may comprise suppressed fragrance particles having a blue hue and the second personal-care composition may comprise suppressed fragrance particles having a red hue. In another embodiment, a first personal-care composition can be a yellow hue and a second personal-care composition can be a green hue, but the first personal-care composition may comprise suppressed fragrance particles having a blue hue and the second personal-care composition may comprise suppressed fragrance particles having a blue hue.

### III. Method of Use

The personal-care article of the present invention may be used to prevent consumer habituation to a particular scent or scents. In a preferred embodiment, the personal-care article is used to sequentially dispense, over time, at least one personal-care product comprising at least two personal-care compositions having different neat and/or suppressed fragrances.

### IV. EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

A. Antiperspirant

### Example 1

### Soft-Solid Antiperspirant Sticks

| | Zone 1 (top | Zone 2 (bottom |
|---|---|---|
| Ingredient | phase), wt % | phase), wt % |
| Cyclopentasiloxane | 18.4617 | 36.5894 |
| Aluminum Zirconium | 8.83 | 17.66 |
| Trichlorohydrex Gly Powder | | |
| Fully Hydrogenated High Erucic | 1.50 | 3.00 |
| Rapeseed Oil | | |
| Dimethicone | 1.67 | 3.33 |
| Petrolatum | 1.00 | 2.00 |
| C18-36 Acid Triglyceride | 0.38 | 0.75 |
| Ppg-14 Butyl Ether | 0.17 | 0.33 |
| Spray-dried Cyclodextrin | | 2.00 |
| Suppressed Fragrance Complex #1 | | |
| Spray-dried Cyclodextrin | 1.00 | |
| Suppressed Fragrance Complex #2 | | |
| Dipropylene Glycol | | 0.50 |
| Neat Fragrance #1 | 0.33 | |
| Neat Fragrance #2 | | 0.50 |
| FD&C Blue #1 | | 0.0006 |
| Total = 100.00 | 33.34 | 66.66 |

Example 1 can be made as follows: First, combine Zone 2 ingredients Cyclopentasiloxane, Dimethicone, C18-36 Acid Triglyceride, PPG-14 Butyl Ether, Petrolatum, & Fully Hydrogenated High Erucic Rapeseed Oil in a suitable container and heat the mixture while stirring to 85° C. Next, add the powders (antiperspirant active, beta cyclodextrin suppressed fragrance complex #2) and mix. The mixture can then be homogenized with a dispersator to fully disperse the powders. Allow to cool to 70° C. Add neat fragrance #2 and then cool the mixture to about 55° C. Lastly, pour the mixture of Zone 2 into antiperspirant canisters. Allow sticks to cool to room temperature.

Next, combine Zone 1 ingredients Cyclopentasiloxane, Dimethicone, C18-36 Acid Triglyceride, PPG-14 Butyl Ether, Petrolatum, & Fully Hydrogenated High Erucic Rapeseed Oil in a suitable container and heat the mixture while stirring to 85° C. Then, add the powders (antiperspirant active, beta cyclodextrin suppressed fragrance complex #1) and mix. The mixture can then be homogenized with a dispersator to fully disperse the powders. Allow to cool to 70° C. Add the neat fragrance #1 and then cool the mixture to about 55° C. Lastly, pour the mixture of Zone 1 on top of Zone 2 in antiperspirant canisters. Allow to cool.

### Examples 2

### Invisible Solid Antiperspirant Sticks

| | Zone 1 (top | Zone 2 (bottom |
|---|---|---|
| Ingredient | phase), wt % | phase), wt % |
| Cyclopentasiloxane | Quantity | Quantity |
| | Sufficient | Sufficient |
| Al Zr Trichlorohydrex Glycinate | 14.50 | 17.50 |
| PPG-14 Butyl Ether | 8.00 | 0 |
| Isopropyl Palmitate | 0 | 10.00 |
| Mineral Oil | 0 | 2.00 |
| Stearyl Alcohol | 12.00 | 12.00 |
| C12-C15 Alkyl Benzoate | 5.00 | 0 |
| Spray Dried Cyclodextrin | 3.00 | |
| Suppressed Fragrance Complex #1 | | |
| Spray Dried Cyclodextrin | | 2.54 |
| Suppressed Fragrance Complex #2 | | |
| Castor Wax | 2.50 | 2.50 |
| Neat Fragrance | 1.25 | 1.25 |
| Fumed Silica | 0.60 | 0.60 |

Example 2 can be made as follows: First, combine Zone 2 waxes (Castor Wax, Stearyl alcohol) with the emollients in a suitable container and heat the mixture while stirring to 75° C. Next, add the powders (antiperspirant active, beta cyclodextrin fragrance complex #2, and silica) and mix. The mixture can then be homogenized with a dispersator to fully disperse the powders. Add the neat fragrance and then cool the mixture to about 60° C. Lastly, pour the cooled mixture of Zone 2 into antiperspirant canisters. Allow sticks to cool to room temperature.

Next, combine Zone 1 waxes (Castor Wax, Stearyl alcohol) with the emollients in a suitable container and heat the mixture while stirring to 75° C. Then, add the powders (antiperspirant active, beta cyclodextrin fragrance complex #1, and silica) and mix. The mixture can then be homogenized with a dispersator to fully disperse the powders. Add the neat fragrance and then cool the mixture to about 60° C. Lastly, pour the cooled mixture of Zone 1 on top of Zone 2 in antiperspirant canisters. Allow sticks to cool to room temperature.

B. Deodorant

### Example 3

### Deodorant Stick

| | Zone 1 (top | Zone 2 (bottom |
|---|---|---|
| Ingredient | phase), wt % | phase), wt % |
| Dipropylene Glycol | Quantity | Quantity |
| | Sufficient | Sufficient |
| Propylene Glycol | 17.80 | 17.80 |
| Water | 21.70 | 21.70 |
| Sodium Stearate | 5.50 | 5.50 |
| Petrolatum Coated Spray | 1.25 | |
| Dried Cyclodextrin | | |
| Fragrance Complex #1 | | |
| Petrolatum Coated Spray | | 1.25 |
| Dried Cyclodextrin | | |
| Fragrance Complex #2 | | |
| Tetra Sodium EDTA | 0.50 | 0.50 |
| PPG-3 Myristyl Ether | 0.04 | 0.04 |
| Neat Fragrance #1 | 3.00 | |
| Neat Fragrance #2 | | 3.00 |

Example 3 can be made as follows: Petrolatum coated cyclodextrin fragrance complexes are prepared by combining spray dried complex particles with petrolatum (e.g., super white protopet manufactured by Witco) at a ratio of 1:1 in a Hamilton Beach custom grind coffee grinder (model 80365). Turn the grinder to the highest speed and mix until the petrolatum fully coats the spray dried cyclodextrin fragrance complex particles. The mixture may have a paste-like consistency. In a suitable vessel, combine all Zone 2 solvents (propylene glycol, dipropylene glycol, water), gellants (sodium stearate, Witconol APM) and tetrasodium EDTA and then heat to 80° C. while mixing. Then add the petrolatum coated complex #2 paste and mix. Cool the mixture to 70° C., and then add neat fragrance #2. Cool the Zone 2 mixture to 65° C. and pour into deodorant canisters.

Next, in a suitable vessel, combine all Zone 1 solvents (propylene glycol, dipropylene glycol, water), gellants (sodium stearate, Witconol APM) and tetrasodium EDTA and then heat to 80° C. while mixing. Then add the petrolatum coated complex #1 paste and mix. Cool the mixture to 70° C., and then add neat fragrance #1. Cool the Zone 1 mixture to 65° C. and pour on top of Zone 2 in deodorant canisters. Allow to cool.

C. Cleansing

### Example 4

### Personal Cleansing Bar

| | Zone 1 (outer | Zone 2 (inner |
|---|---|---|
| Ingredient | phase), wt % | phase), wt % |
| Soap | 80.15 | 80.15 |
| Free Fatty Acid | 5.00 | 5.00 |
| Water | 10.69 | 10.69 |
| Sodium Chloride | 1.11 | 1.11 |
| Titanium Dioxide | 0.25 | 0.25 |
| Spray Dried Cyclodextrin | 2.00 | |
| Fragrance Complex #1 | | |
| Spray Dried Cyclodextrin | | 2.00 |
| Fragrance Complex #2 | | |
| Neat Fragrance | 0.80 | 0.80 |

Example 4 can be made as follows: mix the complexed fragrances and the neat fragrance into dried soap noodles in an amalgamator. Process the material, for example, by milling through a 3-roll soap mill, to obtain a homogenous mixture of perfume and soap flakes. The material is then processed on a plodder and made into a soap bar with Zone 2 contained within Zone 1.

### Example 5

### Personal Cleansing Bar

| | Zone 1 (outer | Zone 2 (inner |
|---|---|---|
| Ingredient | phase), wt % | phase), wt % |
| Soap | 63.26 | 63.26 |
| Free Fatty Acid | 4.29 | 4.29 |
| Water | 7.50 | 7.50 |
| Sodium Chloride | 0.90 | 0.90 |
| Titanium Dioxide | 0.25 | 0.25 |
| Spray Dried Cyclodextrin | 2.00 | 2.00 |
| Fragrance Complex #1 | | |
| Neat Fragrance #1 | 0.80 | |
| Neat Fragrance #2 | | 0.80 |
| Potassium Alkyl Sulfate | 10.00 | 10.00 |
| Sodium Laureth 3 Sulfate | 3.00 | 3.00 |
| Magnesium Silicate | 7.50 | 7.50 |
| Misc. | 0.50 | 0.50 |

Example 5 can be made as follows: mix the complexed fragrance and the neat fragrances into dried soap noodles in an amalgamator. Process the material, for example, by milling through a 3-roll soap mill, to obtain a homogenous mixture of perfume and soap flakes. The material is then processed on a plodder and made into a soap bar with Zone 2 contained within Zone 1.

### Example 6

### Body Wash

| | | Zone 1 (top | Zone 2 (bottom |
|---|---|---|---|
| Ingredient | | phase), wt % | phase), wt % |
| 1. | Water | QS | QS |
| 2. | Sodium Trideceth-3 Sulfate | 6.74 | 6.74 |
| 3. | Sodium Lauryl Sulfate | 6.73 | 6.73 |
| 4. | Sodium Lauroamphoacetate | 3.99 | 3.99 |
| 5. | Disodium EDTA | 0.13 | 0.13 |
| 6. | Sodium Benzoate | 0.17 | 0.17 |
| 7. | Sodium Chloride | 4.00 | 4.00 |
| 8. | PEG-90M | 0.13 | 0.13 |
| | Xanthan Gum | 0.19 | 0.19 |
| | Tridecyl Alcohol | 1.68 | 1.68 |
| | Hydroxypropyl Guar | 0.51 | 0.51 |
| 9. | Citric Acid | 0.8 | 0.8 |
| 10. | Lavender Fragrance | 1.0 | — |
| 10. | Vanilla Fragrance | — | 1.0 |
| 11. | D&C Violet #2 | 0.003 | — |
| 12. | Methyl Chloro Isothiazolinone | 0.0004 | 0.0004 |
| | & Methyl Isothiazolinone | | |

A personal-care article was prepared which contains two compositions with two different fragrances according to the fragrance differentiation method. Two compositions having two different fragrance formulations were prepared by conventional mixing techniques in the order of addition indicated. Addition step 8 containing Tridecyl Alcohol, PEG-90M, Xanthan Gum and Hydroxypropyl Guar was premixed prior to addition to the batch.

The compositions are filled in a tottle with a usable volume of 290 ml. Zone 1 includes the upper 50% (145 ml) of the tottle volume, which has a lavender fragrance. Zone 2 includes the lower 50% (145 ml) of the tottle volume, which has a vanilla fragrance. When the tottle is dispensed at 10 ml per dose, the fragrance changes from vanilla (first 14 days usage) to lavender (second 14 days usage).

Control compositions are filled in a tottle with a usable volume of 290 ml. One control is filled with 290 ml of the Zone 1 composition, which has lavender fragrance. A second control is filled with 290 ml of the Zone 2 composition, which has a vanilla fragrance.

Consumers were then asked to evaluate the product over a usage period of 28 days or completion of the bottle. Questionnaires were used during the 28 day period to evaluate the consumer interest in their product. This data shown in FIG. 12 was graphed to monitor consumer anticipation of a change in fragrance during the usage of the personal-care article of the 290 ml over the 28 days. As shown in FIG. 12, during the period where the consumer experiences a shift from Zone 2 composition to Zone 1 composition, consumers continue to look forward to experiencing the scent versus the single-scented product. This interest continues during the time the product is transitioning and remains directionally higher than the single-scented products.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A personal-care article comprising:
a) a package comprising at least one chamber, a dispensing orifice, a first zone proximate to said dispensing orifice, and a second zone distal to said dispensing orifice, wherein said first zone and said second zone are both located in said at least one chamber; and
b) a personal-care product comprising a first personal-care composition substantially disposed in said first zone and a second personal-care composition substantially disposed in said second zone;
wherein said first composition comprises a first fragrance releaseably associated with a suppressing agent;
wherein said second composition comprises a second fragrance releaseably associated with a suppressing agent; and
wherein said first fragrance is different from said second fragrance.

2) A personal-care article comprising:
a) a package comprising at least one chamber, a dispensing orifice, a first zone proximate to said dispensing orifice, and a second zone distal to said dispensing orifice, wherein said first zone and said second zone are both located in said at least one chamber; and
b) an antiperspirant or deodorant product comprising a first antiperspirant or deodorant composition substantially disposed in said first zone and a second antiperspirant or deodorant composition substantially disposed in said second zone;
wherein said first composition comprises a first antiperspirant active and a first fragrance releaseably associated with a suppressing agent;
wherein said second composition comprises a second antiperspirant active and a second fragrance releaseably associated with a suppressing agent; and
wherein said first fragrance is different from said second fragrance.

3) The personal-care article of claim 2, wherein the concentration of said first antiperspirant active is the same as the concentration of said second antiperspirant active.

4) The personal-care article of claim 2, wherein said first composition further comprises a first neat fragrance and said second composition further comprises a second neat fragrance, wherein said first neat fragrance is the same as said second neat fragrance.

5) The personal-care article of claim 2, wherein said first composition further comprises a first neat fragrance and said second composition further comprises a second neat fragrance, wherein said first neat fragrance and said second neat fragrance are different, wherein said difference is selected from the group consisting of: same chemical make-ups but different concentrations, same concentrations but different chemical make-ups, different chemical make-ups and different concentrations, and combinations thereof.

6) The personal-care article of claim 2, wherein the difference between said first fragrance and said second fragrance is selected from the group consisting of: same chemical make-ups but different concentrations, same concentrations but different chemical make-ups, different chemical make-ups and different concentrations, and combinations thereof.

7) The personal-care article of claim 2, wherein said package comprises a vertical axis and wherein said first zone and said second zone are positioned axially along said vertical axis.

8) The personal-care article of claim 2 wherein said first zone comprises from about 10% to about 90%, by volume, of said package.

9) The personal-care article of claim 2, wherein said first zone comprises from about 30% to about 70%, by volume of said package.

10) The personal-care article of claim 2, wherein said first zone comprises about 50%, by volume of said package.

11) The personal-care article of claim 2, wherein said first composition is substantially dispensed prior to dispensing said second composition.

12) The personal-care article of claim 2, wherein said package comprises a second dispensing orifice, wherein said second dispensing orifice is distal to said first dispensing orifice, wherein said second composition may be dispensed before said first composition is substantially dispensed.

13) The personal-care article of claim 2, wherein said first composition is a different hue from said second composition.

14) The personal-care article of claim 2, wherein said first fragrance is a different hue from said second fragrance.

15) The personal-care article of claim 2, wherein said first zone is in physical contact with said second zone within said package.

16) The personal-care article of claim 2, wherein said package further comprises a third zone between said first zone and said second zone, and wherein said antiperspirant or deodorant product further comprises a third antiperspirant or deodorant composition substantially disposed in said third zone.

17) The personal-care article of claim 16, wherein said third composition comprises a third fragrance, wherein said first fragrance and said second fragrance are different from said third fragrance.

18) The personal-care article of claim 16, wherein said third composition is a different hue from said first composition and said second composition.

19) A personal-care bar product comprising:
a) a horizontal axis, a vertical axis, and a center wherein said horizontal axis and said vertical axis cross;
b) a first zone distal to said center and a second zone proximate to said center; wherein said first zone concentrically surrounds said second zone;
c) a first personal-care composition substantially disposed in said first zone and a second personal-care composition substantially disposed in said second zone;
wherein said first composition comprises a first fragrance releaseably associated with a suppressing agent;
wherein said second composition comprises a second fragrance releaseably associated with a suppressing agent; and
wherein said first fragrance is different from said second fragrance.

20) The personal-care bar product of claim 19, wherein said first composition is a different hue from said second composition.

21) The personal-care bar product of claim 19, wherein said first composition further comprises a first neat fragrance and said second composition further comprises a second neat fragrance, wherein said first neat fragrance is the same as said second neat fragrance.

22) The personal-care article of claim 19, wherein said first composition further comprises a first neat fragrance and said second composition further comprises a second neat fragrance, wherein said first neat fragrance and said second neat fragrance are different, wherein said difference is selected from the group consisting of: same chemical make-ups but different concentrations, same concentrations but different chemical make-ups, different chemical make-ups and different concentrations, and combinations thereof.
